(19) United States
(12) Patent Application Publication
(10) Pub. No.: US 2016/0047111 A1
(43) Pub. Date: Feb. 18, 2016

(54) FAUCET WITH A HIDDEN OZONE DEVICE

(71) Applicant: MU-CHEN TSAI, Taichung City (TW)

(72) Inventor: MU-CHEN TSAI, Taichung City (TW)

(21) Appl. No.: 14/816,468

(22) Filed: Aug. 3, 2015

(30) Foreign Application Priority Data
Aug. 15, 2014 (TW) 103214590

(51) Int. Cl. E03C 1/02; A61L 2/18

(52) U.S. Cl. CPC E03C 1/02; A61L 2/183; E03C 2001/026

(57) ABSTRACT

A faucet with a hidden ozone device has a body and an ozone device. The body has a base tube, an inlet pipe, a connecting tube, an outlet pipe, and a working unit. The connecting tube is mounted on the base tube. The outlet pipe is mounted in the body. The working unit has a dissolving portion and a filter portion. The dissolving portion is mounted in the working unit and is connected with the outlet pipe. The filter portion is connected with the dissolving portion, and has a liquid outlet downward formed on a bottom of the filter portion. The ozone device is connected with the body and has a machine and an ozone pipe. The ozone pipe is connected between the machine and the dissolving portion of the working unit, and is inserted into the base tube and the connecting tube.

### BACKGROUND OF THE INVENTION

1. Field of the Invention

The present invention relates to a faucet, and more particularly to a faucet with a hidden ozone device.

2. Description of Related Art

A conventional ozone device can generate ozone, and the ozone can be dissolved in water. The conventional ozone device can be combined with a conventional faucet. The ozone device has an ozone pipe connected with an outlet of the faucet. The ozone generated from the ozone device can flow in the faucet to be dissolved in water. Then, the water can be applied for cleaning fruits and vegetables to remove pesticide residue on surfaces of fruits and vegetables. Furthermore, ammonia in the water can also be removed. Ozone dissolved in the water can also attack parasitic, viruses, and bacteria in the water.

However, the ozone pipe of the conventional ozone device is mounted on an exterior of the faucet and occupies an additional space. Furthermore, if the ozone is accidentally leaking, the ozone cannot be prevented from filling an indoor space. The ozone is poisonous for a user, such that a risk of ozone poisoning for the user may be increased.

### SUMMARY OF THE INVENTION

The main objective of the present invention is to provide a faucet with a hidden ozone device to resolve the afore-mentioned problems.

The faucet with a hidden ozone device comprises a body and an ozone device.

The body comprises a base tube, a valve, an inlet pipe, a connecting tube, an outlet pipe, a control unit, and a working unit. The valve is mounted in an interior of the base tube, and comprises a pivot formed on a top of the valve and protruding out of a top of the base tube. The inlet pipe communicates with the valve. The connecting tube is mounted on a side of the base tube and communicates with the base tube. The connecting tube comprises a connecting end, a free end, and an outlet portion. The connecting end is connected with the side of the base tube. The free end is opposite the connecting end. The outlet portion is downward formed on a side of the connecting tube, and is adjacent to the free end. The outlet pipe is mounted in the base tube and the connecting tube, communicates with the valve, and is inserted into the outlet portion. The control unit is mounted on the pivot of the valve and is located at the top of the base tube. The working unit is mounted on the outlet portion and comprises a dissolving portion and a filter portion. The dissolving portion is mounted in an upper half of the working unit, is connected with a bottom of the outlet portion, and is connected with the outlet pipe. The filter portion is mounted in a lower half of the working unit, is connected with a bottom of the dissolving portion, and comprises a liquid outlet downward formed on a bottom of the filter portion.

The ozone device is connected with the body and comprises a machine and an ozone pipe. The ozone pipe is connected between the machine and the dissolving portion of working unit, and is inserted into the base tube and the connecting tube.

From the above description, it is noted that the present invention has the following advantages: the ozone pipe is mounted in the base tube and the connecting tube, such that the machine can transport ozone through interiors of the base tube and the connecting tube. The ozone pipe will not occupy an additional space, such that the faucet with a hidden ozone sterilization device can save a storage space effectively and has a neat appearance. Furthermore, the ozone can be prevented from filling an indoor space since the ozone pipe is mounted in the base tube and the connecting tube. When the ozone in the ozone pipe is accidentally leaking, a risk of ozone poisoning for the user can be reduced effectively.

Other objectives, advantages and novel features of the present invention will become more apparent from the following detailed description when taken in conjunction with the accompanying drawings.

### BRIEF DESCRIPTION OF THE DRAWINGS

FIG. 1 is a cross sectional side view in partial section of a preferred embodiment of a faucet with a hidden ozone device in accordance with the present invention; and

FIGS. 2 and 3 are operational side views in partial section of the faucet with a hidden ozone device of FIG. 1.

### DETAILED DESCRIPTION OF PREFERRED EMBODIMENT

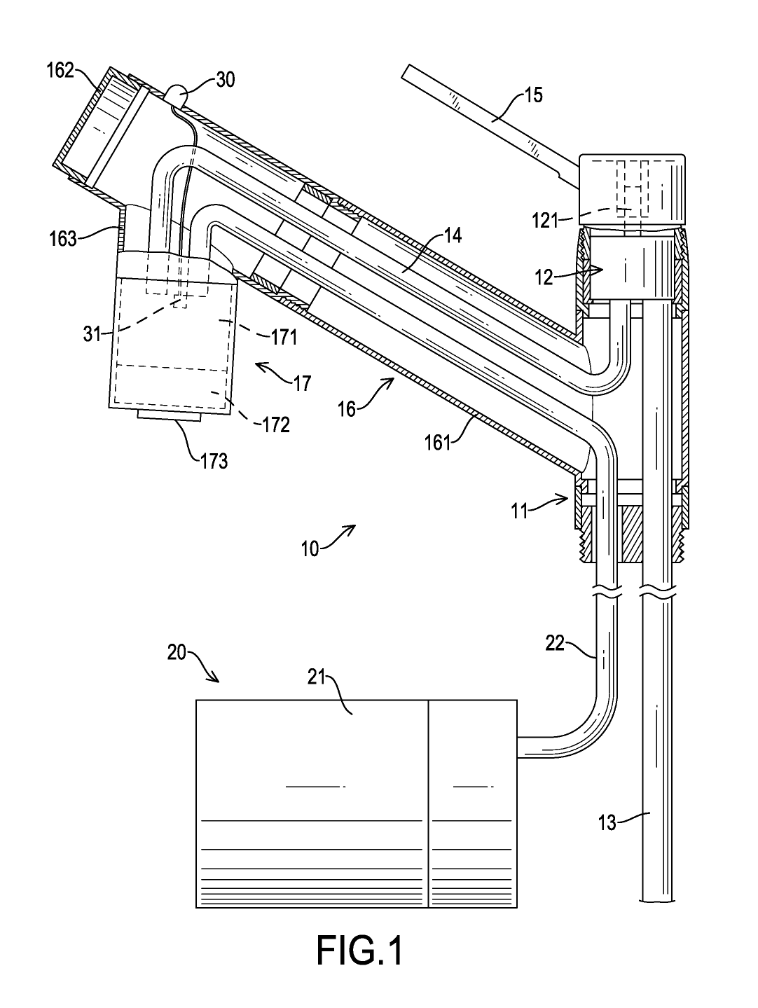

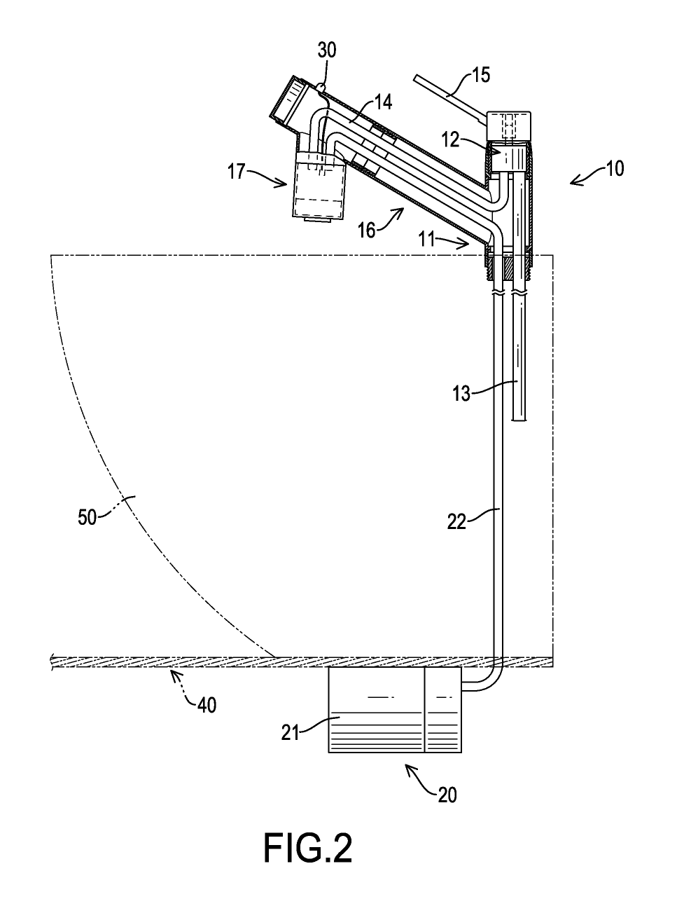

With reference to FIGS. 1 and 2, a preferred embodiment of a faucet with a hidden ozone device in accordance with the present invention comprises a body 10, an ozone device 20, and a caution light 30.

The body 10 comprises a base tube 11, a valve 12, an inlet pipe 13, a connecting tube 16, an outlet pipe 14, a control unit 15, and a working unit 17. The base tube 11 is disposed as an upright tube, and a bottom of the base tube 11 is combined with a wash basin 50. The valve 12 is mounted in an interior of a top of the base tube 11. The valve 12 may be a commercially available valve. The valve 12 comprises an inlet hole and an outlet hole both formed in a bottom of the valve 12. The valve 12 further comprises a pivot 121 formed on a top of the valve 12 and protruding out of the top of the base tube 11. The pivot 121 is applied for controlling the outlet hole to be opened or closed. The inlet pipe 13 is mounted in the base tube 11 and comprises a first end and a second end. The first end of the inlet pipe 13 protrudes out of the base tube 11 and is connected with a liquid source, and the second end of the inlet pipe 13 communicates with the inlet hole of the valve 12.

The connecting tube 16 is mounted on a side of the base tube 11 and communicates with the base tube 11. The connecting tube 16 is inclined relative to the base tube 11 and extends upward. The connecting tube 16 comprises a connecting end 161, a free end 162, and an outlet portion 163. The connecting end 161 is connected with the side of the base tube 11. The free end 162 is opposite the connecting end 161. The outlet portion 163 is downward formed on a side of the connecting tube 16, and is adjacent to the free end 162.

The outlet pipe 14 is mounted in the base tube 11 and the connecting tube 16, and comprises a first end and a second end. The first end of the outlet pipe 14 communicates with the outlet hole of the valve 12. The second end of the outlet pipe 14 is mounted in the outlet portion 163. The control unit 15 is mounted on the pivot 121 of the valve 12 and is located at the top of the base tube 11. The control unit 15 is applied for rotating to control the liquid in the inlet pipe 13 to flow into the outlet pipe 14 through the valve 12.

The working unit 17 is mounted on the outlet portion 163, and comprises a dissolving portion 171 and a filter portion 172. The dissolving portion 171 is mounted in an upper half of the working unit 17, and is connected with a bottom of the outlet portion 163. The second end of the outlet pipe 14 is connected with the dissolving portion 171. The filter portion 172 is mounted in a lower half of the working unit 17, is connected with a bottom of the dissolving portion 171, and comprises a liquid outlet 173 downward formed on a bottom of the filter portion 172.

The ozone device 20 is connected with the body 10 and comprises a machine 21 and an ozone pipe 22. The machine 21 may be formed as a cube. With reference to FIG. 2, the machine 21 can be mounted in a cabinet 40. The ozone pipe 22 is inserted into the base tube 11 and the connecting tube 16, and comprises a first end and a second end. The first end of the ozone pipe 22 is connected with the machine 21. The second end of the ozone pipe 22 is connected with the dissolving portion 171.

The caution light 30 is mounted on the connecting tube 16 and comprises a sensor 31 mounted in the working unit 17 to be adjacent to the second end of the ozone pipe 22 and the second end of the outlet pipe 14. The sensor 31 is applied for sensing whether the dissolving portion 171 of the working unit 17 if full of ozone.

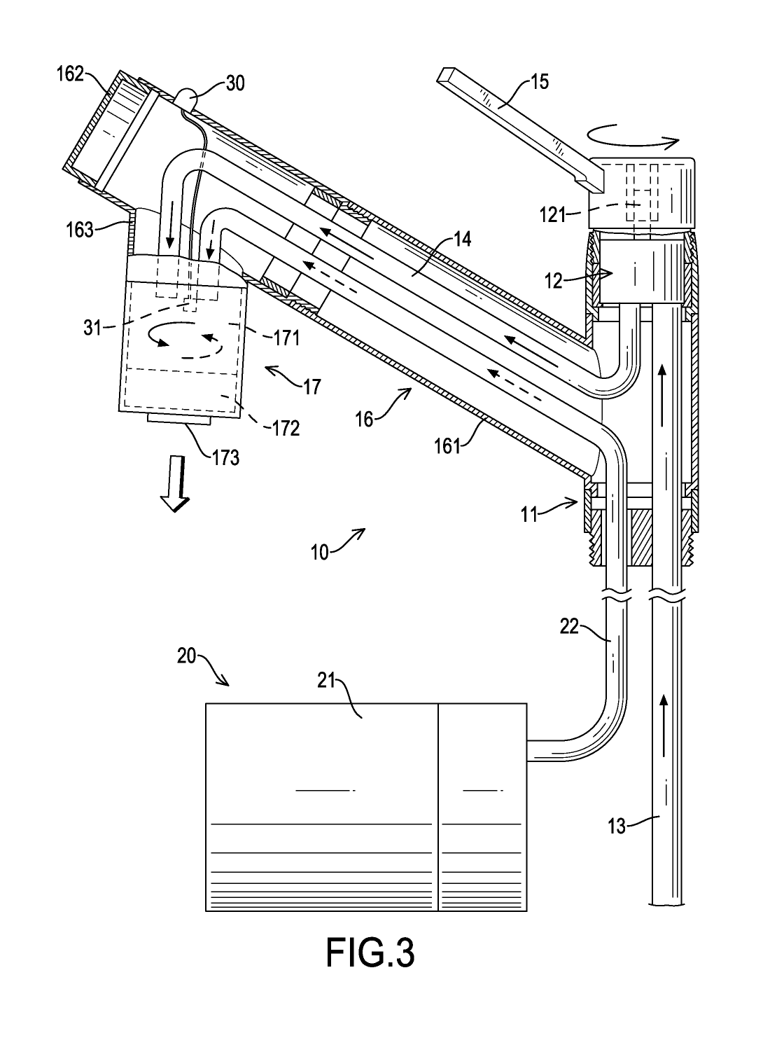

With reference to FIGS. 2 and 3, in use, the base tube 11 is combined with a top of the wash basin 50, and the ozone device 20 is mounted in the cabinet 40 that is mounted on a bottom of the wash basin 50. The first end of the inlet pipe 13 is connected with the liquid source. The control unit 15 can be switched by a user to rotate the pivot 121 to control the outlet hole of the valve 12 to be opened or closed. When the outlet hole of the valve 12 is open, the liquid in the inlet pipe 13 can flow into the outlet pipe 14 through the outlet hole of the valve 12, and then flow into the dissolving portion 171 through the outlet pipe 14. The machine 21 of the ozone device 20 can generate ozone, and the ozone can flow into the dissolving portion 171 through the ozone pipe 22. When the sensor 31 senses that the dissolving portion 171 is full of ozone, the caution light 30 will turn on to remind the user the ozone device 20 is working. After the liquid and the ozone are sufficiently dissolved in the dissolving portion 171, the liquid will flow into the filter portion 172 for filtering away impurities in the liquid. Finally, the liquid will flow out of the liquid outlet 173, wherein the liquid is sterilized by the ozone, and is filtered by the filter portion 172. Compared with a conventional faucet combined with a conventional ozone device that occupies an additional space, the faucet with a hidden ozone device in accordance with the present invention can save a storage space effectively since the ozone pipe 22 is mounted in the base tube 11 and the connecting tube 16. Furthermore, when the ozone in the ozone pipe 22 is accidentally leaking, the ozone can be prevented from filling an indoor space since the ozone pipe 22 is mounted in the base tube 11, the connecting tube 16, the wash basin 50, and the cabinet 40. Therefore, when the ozone in the ozone pipe 22 is accidentally leaking, a risk of ozone poisoning for the user can be reduced effectively.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A faucet with a hidden ozone device comprising:
a body comprising
a base tube;
a valve mounted in an interior of the base tube, and comprising a pivot formed on a top of the valve and protruding out of a top of the base tube;
an inlet pipe communicating with the valve;
a connecting tube mounted on a side of the base tube and communicating with the base tube, and comprising
a connecting end connected with the side of the base tube;
a free end being opposite the connecting end; and
an outlet portion downward formed on a side of the connecting tube, and being adjacent to the free end;
an outlet pipe mounted in the base tube and the connecting tube, communicating with the valve, and inserted into the outlet portion;
a control unit mounted on the pivot of the valve and located at the top of the base tube; and
a working unit mounted on the outlet portion and comprising
a dissolving portion mounted in an upper half of the working unit, connected with a bottom of the outlet portion, and connected with the outlet pipe; and
a filter portion mounted in a lower half of the working unit, connected with a bottom of the dissolving portion, and comprising a liquid outlet downward formed on a bottom of the filter portion; and
an ozone device connected with the body and comprising
a machine; and
an ozone pipe connected between the machine and the dissolving portion of the working unit, and inserted into the base tube and the connecting tube.

2. The faucet with a hidden ozone device as claimed in claim 1 further comprising a caution light mounted on the connecting tube and comprising a sensor mounted in the working unit to be adjacent to the ozone pipe and the outlet pipe.
